(19) United States
(12) Patent Application Publication
(10) Pub. No.: US 2026/0345159 A1
(43) Pub. Date: Nov. 5, 2026

(54) MEDICAL STAPLER WITH FORKED GUIDING PLATE

(71) Applicant: Hamid Ayub, Plantation, FL (US)

(72) Inventor: Hamid Ayub, Plantation, FL (US)

(21) Appl. No.: 19/633,013

(22) Filed: Mar. 30, 2026

Related U.S. Application Data
(63) Continuation-in-part of application No. 19/198,376, filed on May 5, 2025, now Pat. No. 12,678,159.

(51) Int. Cl. A61B 17/072

(52) U.S. Cl. CPC A61B 17/07207

(57) ABSTRACT

The present invention discloses a medical stapler for wound and incision closure comprising a paired staple assembly and a forked guiding plate. The paired staple assembly comprises a standard staple having a pair of standard legs for deep tissue engagement and a fine staple having a pair of fine legs for superficial skin engagement, arranged side by side. The standard and fine staples may be two separate components or a single integrated unit. A forked guiding plate defines a first crimping formation configured to assist in deforming the standard legs inward and a second crimping formation configured to assist in deforming the fine legs inward, the two formations being laterally offset corresponding to the side-by-side staple arrangement. Both staples are simultaneously deployed through the guiding plate in a single firing cycle. A staple strip comprising a plurality of paired staple sets enables sequential dispensing.

### CROSS-REFERENCE TO RELATED APPLICATION

This application is a Continuation-in-Part (CIP) of U.S. patent application Ser. No. 19/198,376 filed on May 5, 2025, which is a Continuation-in-Part (CIP) of U.S. patent application Ser. No. 18/945,770, filed on Nov. 11, 2024 which is a Continuation-in-Part (CIP) of U.S. Ser. No. 12/089,840B 2 , filed on 11 Feb. 2024 and issued Sep. 17, 2024, the disclosure of which is hereby incorporated by reference in its entirety in this application.

### FIELD OF INVENTION

Embodiments of a present disclosure relate to the field of surgical instruments and more particularly to a surgical stapler for approximation of skin and deeper tissues.

### BACKGROUND

Conventional surgical skin staples typically have two spikes or legs and are generally U-shaped. After being deployed, such staples form a rectangular shape, which may turn within the skin, or may buckle. This deployment can lead to overlapping or misalignment of the wound edges, potentially causing unpleasant cosmetic outcomes.

Four-spike staple configurations having two longer outer spikes and two shorter inner spikes have been proposed, wherein the shorter inner spikes serve to stabilize the skin and help prevent overlapping of the wound edges. However, systems incorporating such staples may also require additional stabilizing components that extend beneath the skin surface. Upon removal, these components may necessitate manual suturing or be left open, which can result in patient discomfort, potentially increased healing time, or aesthetically unfavourable scarring.

To address the concerns in the background, an effective way for wound closures is required.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

The present invention provides a medical stapler for wound and incision closure comprising a paired staple assembly and a forked guiding plate. The paired staple assembly comprises a standard staple having a pair of standard legs configured for deep tissue engagement, and a fine staple having a pair of fine legs configured for superficial skin engagement, the standard staple and the fine staple being arranged side by side. The standard staple and the fine staple may be formed as two physically separate components or as a single integrated unit.

The forked guiding plate defines a first crimping formation configured to assist in crimping the standard legs inward, and a second crimping formation configured to assist in crimping the fine legs inward. The first and second crimping formations are laterally offset from one another in a side-by-side arrangement corresponding to the side-by-side arrangement of the standard staple and the fine staple. Upon actuation of a driver, both the standard staple and the fine staple are deployed simultaneously through the respective crimping formations in a single firing cycle, achieving multi-depth wound closure.

An object of the present invention is to provide a guiding plate architecture wherein each leg type is crimped by a dedicated formation, thereby achieving controlled deformation of both standard and fine legs independently of one another.

A further object is to provide a staple strip assembly comprising a plurality of paired staple sets arranged for sequential dispensing, each set comprising one standard staple and one fine staple arranged side by side.

A further object of the present invention is to expedite the wound closure process during surgical procedures, thereby significantly reducing the time required to complete skin and tissue approximation. This, in turn, minimizes the overall duration for which the patient is maintained under anaesthesia, contributing to improved surgical efficiency and reduced anaesthesia-related risks.

### BRIEF DESCRIPTION OF DRAWINGS

The disclosure will be described and explained with additional specificity and detail with the accompanying figures in which:

FIG. 1 is a perspective view of a medical stapler (100) in accordance with an embodiment of the present disclosure.

FIG. 2A is a perspective view of a single paired staple set (200) comprising a standard staple (202) and a fine staple (204) arranged side by side.

FIG. 2B is a perspective view of a staple strip (112) comprising a plurality of paired staple sets (200) arranged for sequential dispensing, each set comprising a standard staple (202) and a fine staple (204) side by side.

FIG. 3A is an isometric view of the forked guiding plate (102) with the staple strip (112) loaded thereon, showing the first crimping formation (114) configured to assist in crimping the standard legs and the second crimping formation (116) configured to assist in crimping the fine legs.

FIG. 3B is an isometric view of the deployment assembly showing the driver (104), the forked guiding plate (102), the standard staple (202) and the fine staple (204) with the standard legs (206a, 206b) and fine legs (208a, 208b) extending below the guiding plate.

FIG. 4A is a cross-sectional view showing the paired staple set (200) positioned for insertion, with the driver (104) above, the forked guiding plate (102) below, and the skin (300) and tissue (302) layers beneath.

FIG. 4B is a cross-sectional view showing the paired staple set (200) after insertion, with the standard legs (206a, 206b) crimped inward in the tissue layer (302) and the fine legs (208a, 208b) crimped inward in the skin layer (300).

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

The present disclosure provides a medical stapler for wound and incision closure comprising a paired staple assembly and a forked guiding plate. The paired staple assembly comprises a standard staple (202) having a pair of standard legs (206a, 206b) and a fine staple (204) having a pair of fine legs (208a, 208b), the standard staple and the fine staple being arranged side by side. In this side-by-side arrangement, the legs of the standard staple and the legs of the fine staple are positioned laterally adjacent to one another. The side-by-side arrangement enables each staple to engage a corresponding dedicated crimping formation on the forked guiding plate (102).

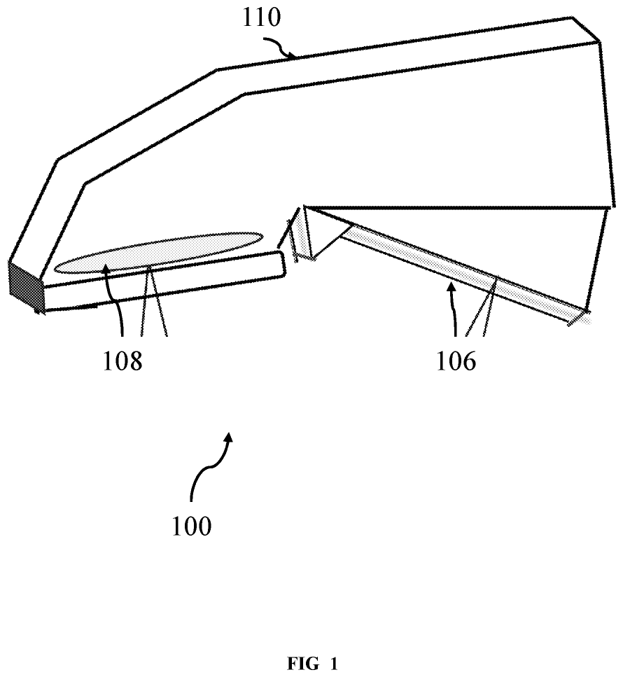

Referring to FIG. 1, a medical stapler (100) in accordance with the present disclosure comprises an enclosed body (110), a firing handle (106) operatively coupled to the body (110), a staple count indicator (108) disposed on the body (110), a driver (104) disposed within the body (110), and a forked guiding plate (102) disposed at a deployment end of the body (110).

The enclosed body (110) defines an internal magazine configured to receive and retain a staple strip (112). The firing handle (106) is pivotally connected to the body (110) and, upon manual actuation by a surgeon, drives the driver (104) in a deployment direction toward the forked guiding plate (102). The staple count indicator (108) provides a visual indication of the number of paired staple sets remaining in the staple strip (112).

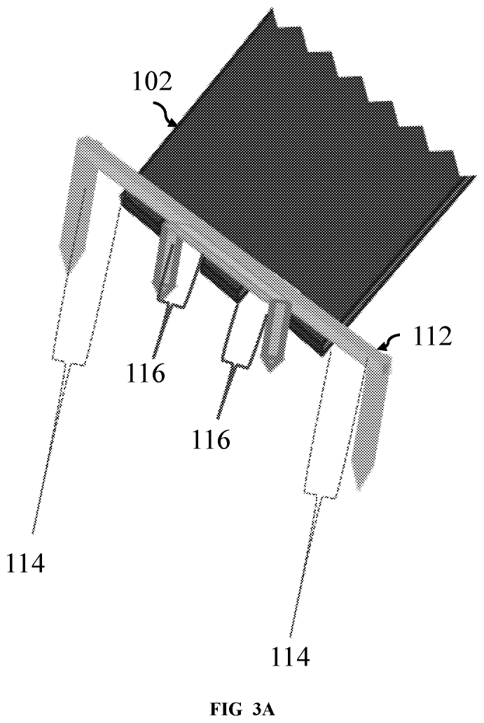

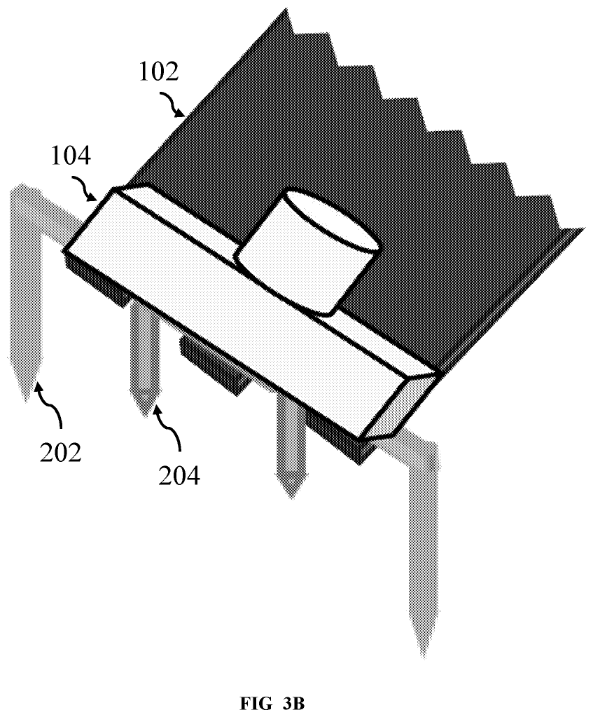

As shown in FIG. 3B, the driver (104) is positioned above the staple strip (112), and the forked guiding plate (102) is positioned below the staple strip (112). This arrangement defines the deployment axis along which the driver (104) advances a paired staple set through the guiding plate (102) and into tissue.

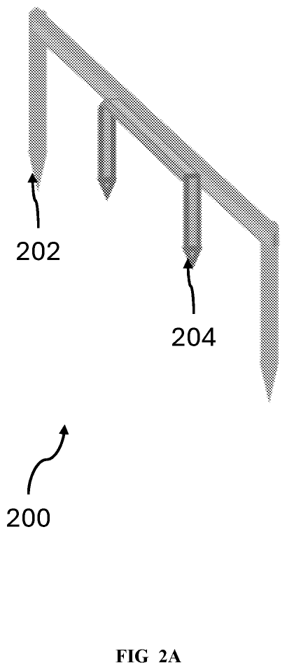

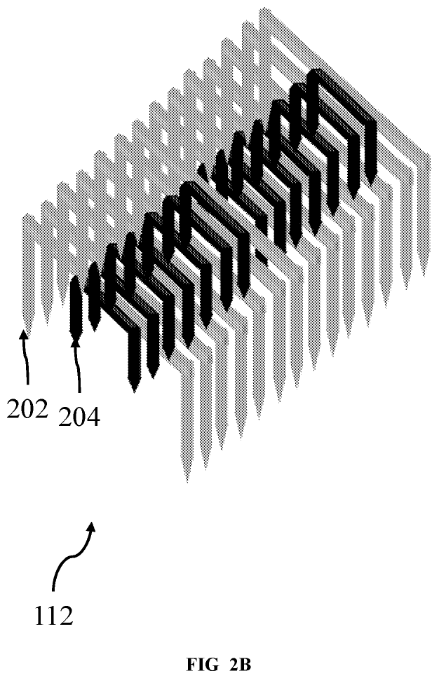

Referring to FIG. 2A, a single paired staple set (200) comprises a standard staple (202) and a fine staple (204) arranged side by side.

The standard staple (202) comprises an arch portion (210) and a pair of standard legs (206a, 206b) extending downward from the arch portion (210). Each standard leg (206a, 206b) has a first length configured for penetrating through both the skin layer (300) and the underlying tissue layer (302) to achieve deep tissue anchoring. The standard legs (206a, 206b) are oriented to penetrate tissue at an angle of approximately 90 degrees±15 degrees relative to the tissue surface.

The fine staple (204) comprises an arch portion (212) and a pair of fine legs (208a, 208b) extending downward from the arch portion (212). Each fine leg (208a, 208b) has a second length, shorter than the first length, configured for engaging the superficial skin layer (300) without penetrating into the deeper tissue layer (302). The fine legs (208a, 208b) are oriented to penetrate tissue at an angle of approximately 90 degrees±15 degrees relative to the tissue surface.

The standard staple (202) and the fine staple (204) are positioned adjacent to one another in a side-by-side arrangement, such that the arch portions (210, 212) lie substantially in the same plane and the standard legs (206a, 206b) and the fine legs (208a, 208b) extend downward in the same deployment direction.

In a first embodiment, the standard staple (202) and the fine staple (204) are two physically separate components. Each is independently formed from biocompatible wire stock and the two components are loaded side by side into the magazine of the medical stapler (100). This two-part configuration permits the standard staple and the fine staple to be manufactured from different wire gauges, materials, or surface treatments as may be required for their respective tissue-engagement functions. The concept of separable fine and standard leg components, as contemplated in the parent applications in the context of independent deployment capability, is here implemented as physically discrete staple units arranged side by side from the point of manufacture.

In a second embodiment, the standard staple and the fine staple are formed as a single integrated unit (200). The integrated unit (200′) comprises a continuous structure from which both the standard legs (206a, 206b) and the fine legs (208a, 208b) extend downward side by side. The integrated unit (200′) is formed from a single continuous length of biocompatible wire stock shaped into the side-by-side configuration. The integrated unit (200′) provides the same side-by-side leg arrangement as the two-part embodiment while ensuring that the relative positioning of the standard legs and fine legs is fixed during manufacturing and deployment.

In both embodiments, the standard staple and the fine staple (or the standard-leg portion and the fine-leg portion of the integrated unit) are configured for simultaneous deployment by the driver (104) through the forked guiding plate (102).

Referring to FIG. 2B, the staple strip (112) comprises a plurality of paired staple sets (200) arranged in a linear array for sequential dispensing from the magazine of the medical stapler (100). Each paired staple set (200) in the strip comprises one standard staple (202) and one fine staple (204) positioned side by side.

Adjacent paired staple sets (200) in the strip (112) are connected at their respective arch portions. Upon deployment of an individual paired staple set, the deployment force separates the deployed pair from the remainder of the strip (112). The strip (112) advances incrementally after each firing cycle, positioning the next paired staple set for deployment. The staple count indicator (108) is operatively coupled to the strip advancement mechanism to provide a visual indication of remaining paired staple sets.

Referring to FIG. 3A, the forked guiding plate (102) is disposed at the deployment end of the medical stapler (100) and is positioned opposite the driver (104), with the staple strip (112) interposed therebetween.

The guiding plate (102) defines a crimping surface facing toward the driver (104) and the staple strip (112). The crimping surface has formed therein a first crimping formation (114) and a second crimping formation (116). The first and second crimping formations are laterally offset from one another in a side-by-side arrangement corresponding to the side-by-side arrangement of the standard staple (202) and the fine staple (204) in the paired staple set (200).

The first crimping formation (114) is designed to assist in the crimping action of the standard legs (206a, 206b) of the standard staple (202). The first crimping formation (114) is configured to receive the standard legs during deployment and to assist in deforming the standard legs inward, thereby achieving deep tissue anchoring in the tissue layer (302).

The second crimping formation (116) is designed to assist in the crimping action of the fine legs (208a, 208b) of the fine staple (204). The second crimping formation (116) is configured to receive the fine legs during deployment and to assist in deforming the fine legs inward, thereby achieving superficial skin approximation in the skin layer (300).

The term “forked” as used herein refers to the guiding plate (102) having at least two laterally separated crimping formations (114, 116) on the same plate body, each formation independently configured for a different staple type. The forked architecture ensures that each leg type encounters its corresponding crimping formation during deployment, enabling both the standard staple and the fine staple to be crimped by their respective dedicated formations in a single firing cycle.

The guiding plate (102) is preferably formed from a biocompatible material of sufficient hardness to effect deformation of the staple legs upon deployment. Suitable materials include surgical-grade stainless steel, hardened tool steel, or cobalt-chromium alloy. In single-use disposable embodiments, the guiding plate (102) may be formed from medical-grade polymer with metallic inserts at the crimping formations.

The standard staple (202) and the fine staple (204) are each preferably formed from biocompatible materials including surgical-grade stainless steel, titanium, titanium alloys, biocompatible metals or absorbable material that are selected for their mechanical strength, corrosion resistance, and compatibility with human tissue.

In further embodiments, other variations of the crimping formation configuration may be utilized, provided they maintain the functional objective of dedicated crimping for each leg type. It is understood that in other embodiments, variations in the leg dimensions may be employed, such as legs with different lengths or thicknesses, to accommodate specific wound types or surgical requirements, while maintaining the side-by-side arrangement and the forked guiding plate architecture.

In a further embodiment, the forked guiding plate (102) may be removably mounted to the body (110) of the medical stapler (100), permitting the guiding plate to be replaced with a plate having different crimping formation configurations suited to different staple sizes or clinical applications.

Referring to FIG. 3B, the driver (104) is a substantially rigid member disposed within the body (110) of the medical stapler (100) above the staple strip (112). The driver (104) has a lower deployment surface configured to contact the arch portions (210, 212) of a paired staple set (200) and to transmit the deployment force generated by actuation of the firing handle (106). The deployment surface of the driver (104) has a lateral width sufficient to span both the standard staple (202) and the fine staple (204) in the side-by-side arrangement, ensuring that both staples are driven simultaneously.

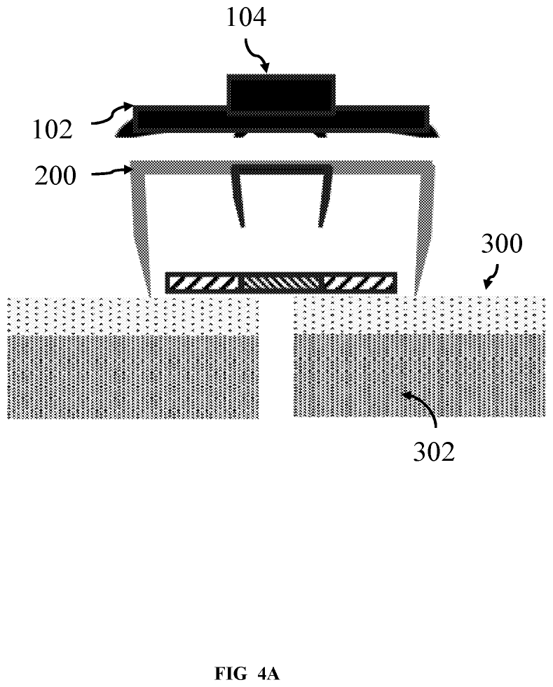

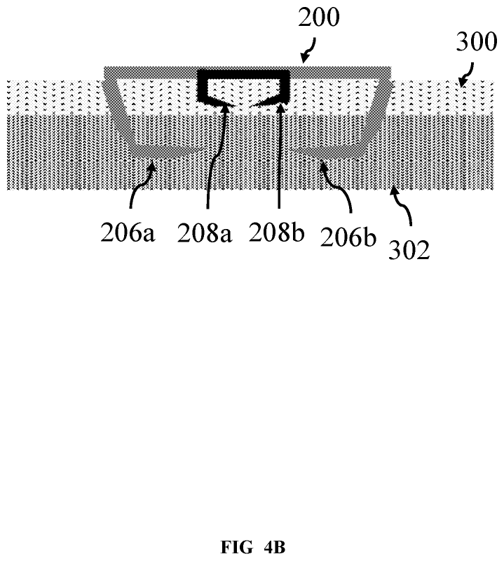

Referring to FIGS. 4A and 4B, in a pre-insertion configuration (FIG. 4A), the medical stapler (100) is positioned with the forked guiding plate (102) placed against the skin surface (300) at the wound site. The driver (104) is in a retracted position above the staple strip (112). A paired staple set (200) is positioned at the deployment station, with the standard legs (206a, 206b) aligned with the first crimping formation (114) and the fine legs (208a, 208b) aligned with the second crimping formation (116).

Upon actuation of the firing handle (106), the driver (104) descends and contacts the arch portions (210, 212) of the paired staple set (200). The driver (104) simultaneously drives both the standard staple (202) and the fine staple (204) downward through the forked guiding plate (102) and into the tissue.

During deployment, the standard legs (206a, 206b) engage the first crimping formation (114) of the guiding plate (102). The first crimping formation (114) assists in deforming the standard legs inward as they penetrate through the skin layer (300) and into the tissue layer (302). The standard legs (206a, 206b) are crimped into an inward-bent configuration within the tissue layer (302), providing deep tissue anchoring.

Simultaneously, the fine legs (208a, 208b) engage the second crimping formation (116) of the guiding plate (102). The second crimping formation (116) assists in deforming the fine legs inward as they penetrate into the skin layer (300). The fine legs (208a, 208b) are crimped into an inward-bent configuration within the skin layer (300), providing superficial skin approximation.

In the post-insertion configuration (FIG. 4B), the medical stapler (100) is withdrawn from the wound site, leaving the crimped paired staple set (200) in situ. The arch portions (210, 212) of the standard staple (202) and fine staple (204) remain at or slightly above the skin surface (300), spanning the wound. The standard legs (206a, 206b) are crimped inward within the tissue layer (302), and the fine legs (208a, 208b) are crimped inward within the skin layer (300). Together, the crimped standard staple and crimped fine staple achieve multi-depth wound closure—deep tissue anchoring and superficial skin approximation—in a single deployment cycle.

The advantages of the described medical stapler invention include:
a. Dedicated Crimping Per Leg Type: Each leg type—standard and fine—is crimped by a formation on the guiding plate dedicated to that leg type. The forked architecture ensures that the crimping action is appropriate to each leg's length and intended engagement depth.
b. Simultaneous Multi-Depth Closure: The side-by-side staple arrangement and forked plate enable deployment and crimping of both standard and fine staples in a single firing cycle, achieving multi-depth wound closure without the need for multiple separate staple applications.
c. Manufacturing Flexibility: The two-part embodiment permits independent selection of wire gauge, material, and surface treatment for each staple type. The integrated embodiment simplifies handling and loading while maintaining the side-by-side leg arrangement.
d. Compatibility with Strip Dispensing: The paired staple sets are arranged in a linear strip (112) for sequential dispensing, compatible with single-track magazine architectures.
e. Reduced Operative Time: By achieving deep tissue anchoring and superficial skin approximation in a single deployment cycle, the present invention reduces the total number of stapler actuations required, reduces overall operative time, and minimizes the duration for which the patient is maintained under anaesthesia.
f. Even Pressure Distribution: The side-by-side arch arrangement distributes compressive forces across the wound site at two depths simultaneously, promoting uniform tissue approximation and reducing the risk of wound dehiscence.
g. Rotational Stability: The paired staple configuration, with four total legs engaging tissue at two depths, provides enhanced resistance to rotational displacement relative to a single two-legged staple.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present disclosure are intended to be illustrative, but not limiting, of the scope of the invention, which is set forth in the following claims.

What is claimed is:

1. A medical stapler for wound closure, comprising:
a paired staple assembly comprising a standard staple having a pair of standard legs of a first length and a fine staple having a pair of fine legs of a second length shorter than the first length, the standard staple and the fine staple being arranged side by side;
a driver configured to advance the paired staple assembly in a deployment direction;
and a forked guiding plate defining:
a first crimping formation configured to assist in deforming the standard legs inward upon deployment; and
a second crimping formation laterally offset from the first crimping formation and configured to assist in deforming the fine legs inward upon deployment;
wherein the driver is configured to simultaneously deploy both the standard staple and the fine staple through the respective first and second crimping formations in a single firing cycle.

2. The medical stapler of claim 1, wherein each of the standard legs and the fine legs is oriented to penetrate tissue at an angle of approximately 90 degrees±15 degrees relative to the tissue surface.

3. The medical stapler of claim 1, wherein the standard staple and the fine staple are two physically separate components loaded side by side in the medical stapler.

4. The medical stapler of claim 1, wherein the standard staple and the fine staple are formed as a single integrated unit with the standard legs and the fine legs extending side by side from a continuous structure.

5. The medical stapler of claim 1, further comprising a staple strip comprising a plurality of paired staple assemblies arranged in a linear array for sequential dispensing, each paired staple assembly comprising a standard staple and a fine staple arranged side by side.

6. The medical stapler of claim 1, further comprising a staple count indicator operatively coupled to a strip advancement mechanism to indicate remaining paired staple assemblies.

7. The medical stapler of claim 1, wherein the standard staple and the fine staple are formed from a biocompatible material selected from the group consisting of surgical-grade stainless steel, titanium, titanium alloy, and bio-absorbable material.

8. The medical stapler of claim 1, wherein the forked guiding plate is formed from a material having a hardness sufficient to effect deformation of the standard legs and the fine legs upon deployment.

9. The medical stapler of claim 1, wherein the standard legs are crimped inward in a tissue layer and the fine legs are crimped inward in a skin layer to achieve multi-depth wound closure.

10. A forked guiding plate for use with a medical stapler configured to deploy a paired staple assembly comprising a standard staple and a fine staple arranged side by side, the forked guiding plate comprising:
a plate body having a crimping surface;
a first crimping formation defined in the crimping surface, the first crimping formation configured to assist in deforming standard legs of the standard staple inward upon deployment; and
a second crimping formation defined in the crimping surface and laterally offset from the first crimping formation, the second crimping formation configured to assist in deforming fine legs of the fine staple inward upon deployment;
wherein the first and second crimping formations are arranged side by side on the plate body corresponding to the side-by-side arrangement of the standard staple and the fine staple.

11. The forked guiding plate of claim 10, wherein the plate body is formed from a biocompatible material having a hardness sufficient to effect deformation of the standard legs and the fine legs.

12. A method of closing a wound, comprising:
positioning a forked guiding plate of a medical stapler adjacent a wound site;
simultaneously deploying a standard staple and a fine staple arranged side by side through the forked guiding plate using a driver, the standard staple having a pair of standard legs of a first length and the fine staple having a pair of fine legs of a second length shorter than the first length;
crimping the standard legs inward by engagement with a first crimping formation of the forked guiding plate to anchor the standard legs in a tissue layer; and
crimping the fine legs inward by engagement with a second crimping formation of the forked guiding plate to approximate the fine legs in a skin layer;
thereby achieving multi-depth wound closure in a single deployment cycle.

13. The method of claim 12, wherein the standard staple and the fine staple are two physically separate components deployed simultaneously by the driver.

14. The method of claim 12, wherein the standard staple and the fine staple are formed as a single integrated unit.
